(19) United States
(12) Patent Application Publication
(10) Pub. No.: US 2026/0125533 A1
(43) Pub. Date: May 7, 2026

(54) PLASTIC DEPOLYMERIZATION USING METAL ORGANIC FRAMEWORK BASED CATALYSTS

(71) Applicant: BASELL POLIOLEFINE ITALIA S.R,I., Milano (IT)

(72) Inventors: Diego Brita, Ferrara (IT); Simona Guidotti, Ferrara (IT); Dario Liguori, Ferrara (IT); Francesco Menichelli, Ferrara (IT)

(73) Assignee: BASELL POLIOLEFINE ITALIA S.R,I., Milano (IT)

(21) Appl. No.: 19/117,568

(22) PCT Filed: Oct. 5, 2023
(86) PCT No.: PCT/EP2023/077544

§ 371 Date: Apr. 1, 2025

(30) Foreign Application Priority Data
Oct. 7, 2022 (EP) 22200229.7

(51) Int. Cl. C08J 11/26; B01J 31/16

(52) U.S. Cl. CPC C08J 11/26; B01J 31/1691; B01J 2231/005; B01J 2531/16; B01J 2531/26; B01J 2531/48; B01J 2531/842; C08J 2323/06; C08J 2323/12; C08J 2423/06; C08J 2423/12

(57) ABSTRACT

A process for depolymerizing plastic waste, including the steps of: a) providing a melt plastic waste feedstock made from or containing recycled polypropylene and b) subjecting the melt product obtained in (a) to a temperature ranging from 280° C. to 600° C., thereby obtaining a depolymerization product; wherein the melt product, the depolymerization product, or both are contacted with a metal organic framework (MOF) catalyst.

### FIELD OF THE DISCLOSURE

In general, the present disclosure relates to the field of chemistry. More specifically, the present disclosure relates to polymer chemistry. In particular, the present disclosure relates to a catalytic method for depolymerizing plastic feedstock and to a catalyst for the depolymerization.

### BACKGROUND OF THE DISCLOSURE

For some applications, plastics are inexpensive and durable materials for manufacturing a variety of products. As such, the production of plastics has increased dramatically over the last decades. In some instances, the durability of the plastics contributes to an increase in the amount of plastics are filling up landfill sites and natural habitats. In some instances, degradable and biodegradable plastics persist depending on environmental factors, such as ultraviolet light exposure, temperature, ant the presence of microorganisms.

Currently, plastic recycling primarily includes mechanical recycling and chemical recycling. With mechanical recycling, plastics are transformed without changing their chemical structure and may be used to produce new materials. In some instances, mechanical recycling steps include: collecting plastic wastes; sorting plastic by type and color; pressing or milling plastics for packaging plastics; washing and drying plastics; and reprocessing plastics into pellets by agglutinating, extruding and cooling the plastics. In some instances, the plastics are polyolefins such as polyethylene (PE) and polypropylene (PP).

Alternatively, chemical recycling modifies the structure of plastics, thereby permitting use of the resulting plastics as raw material for different industries or as a basic input or feedstock for manufacturing new plastic products. In some instances, chemical recycling steps include: collecting plastics and heating the plastics to a temperature at which the polymers break down into fragments. In some instances, this process is referred to as depolymerization and converts plastic waste material to liquid fuel by thermal degradation (cracking) in the absence of oxygen. In some instances, plastics waste is first melted within a stainless steel chamber, under an inert purging gas. Next, the molten material is heated to a gaseous state. Then, the gaseous material is drawn and condensed in one or more condensers, thereby yielding a hydrocarbon distillate made from or containing straight and branched chain aliphatic, cyclic aliphatic, and aromatic hydrocarbons. In some instances, the resulting mixture is used as a fuel. In some instances, the resulting mixture is used as a feedstock in a thermocatalytic process for obtaining refined chemicals such as monomers.

### SUMMARY OF THE DISCLOSURE

In a general embodiment, the present disclosure provides a process for depolymerizing plastics including the steps of:
a) providing a melt plastic waste feedstock made from or containing recycled polypropylene; and
b) subjecting the melt product obtained in (a) to a temperature ranging from 280° C. to 600° C., thereby obtaining a depolymerization product;
wherein the melt product, the depolymerization product, or both are contacted with a metal organic framework (MOF) catalyst.

In some embodiments, the amount of catalyst used ranges from 0.1 to 20 wt. %, alternatively 0.1-10 wt. %, alternatively from 0.1 to 5 wt. %, with respect to the total weight of plastic waste feedstock and catalyst.

In some embodiments, the plastic waste feedstock is made from or containing a mixture of polyethylene and polypropylene in a weight ratio 85:15 to 15:85, alternatively 80:20 to 20:80. In some embodiments, the polyethylene is selected from the group consisting of high density polyethylene (HDPE), low-density polyethylene (LDPE), and linear low density polyethylene (LLDPE). In some embodiments, the polypropylene (PP) is a propylene homopolymer or a propylene copolymer, having a lower amount of ethylene, butene, or both. In some embodiments, the feedstock is made from or containing other polyolefins like polybutene. In some embodiments, the feedstock is further made from or containing other polymeric materials. In some embodiments, the other polymeric materials are selected from the group consisting of polystyrene (PS), ethyl-vinyl acetate copolymer (EVA), ethyl-vinyl alcohol copolymer (EVOH), polyvinyl chloride (PVC), and mixtures thereof. In some embodiments, the feedstock is made from or containing more than 80% wt of a mixture between polyethylene and polypropylene, wherein polypropylene accounts for more than 50% wt of the polypropylene/polyethylene mixture.

In some embodiments, the depolymerization process occurs in an oxygen-free environment. In some embodiments, an oxygen containing atmosphere is not introduced into the depolymerization system. In some embodiments, the barrier to the potentially oxygen-containing atmosphere is obtained with a series of expedients. In some embodiments, the series of expedients is selected from the group consisting of nitrogen blanketing and a vacuum system connected to a barrel of the extruder.

In some embodiments, the plastic feedstock mixture is charged into the feeding system of the depolymerization reactor by a hopper, or two or more hoppers in parallel. In some embodiments, the oxygen present in the atmosphere of the plastic waste material is eliminated inside the hopper(s).

In some embodiments, the plastic feedstock is fed directly into the depolymerization reactor for small scale tests. In some embodiments and for larger scale, the plastic feedstock is fed to the depolymerization reactor by an extruder.

In some embodiments, the plastic scrap is melted and then injected into the depolymerization reactor. In some embodiments, the extruder receives the plastic scrap cut in small pieces into the feed hopper, conveys the stream in the melting section, and heats the polymer by combined action of mixing energy and heat supplied by barrel heaters. In some embodiment, the melting temperature ranges from 250° C. to 350° C.

In some embodiments, additives are incorporated in the melt. In some embodiments, the additives reduce corrosivity of plastic scrap or improve depolymerization efficiency.

In some embodiments and during the extrusion, one or more degassing steps remove residual humidity present in the product.

In some embodiments and before being fed to the reactor, the melt stream is filtered, thereby removing solid impurities from the plastic waste.

In some embodiments, the extrusion systems are selected from the group consisting of single screw extruders, twin screw extruders, twin screw extruders with gear pump, and combinations thereof.

In some embodiments, mixing of the plastic waste feedstock and catalyst takes place directly into the depolymerization reactor or beforehand outside the reactor. In some embodiments, the mixing takes place in the depolymerization reactor, wherein the catalyst is fed (a) by directly pouring the solid catalyst into the reactor under a nitrogen atmosphere or (b) in a form of a liquid hydrocarbon slurry or a semisolid paste using dedicated devices. In some embodiments and in small scale systems, the catalyst is fed by directly pouring the solid catalyst into the reactor under a nitrogen atmosphere.

In some embodiments, the mixing takes place outside the depolymerization reactor. In some embodiments, the mixing takes place outside the depolymerization reactor, wherein the catalyst is mixed with plastic scrap in a homogenizer apparatus and the mixture is then pelletized. In some embodiments, the pellets are further made from or containing other additives. In some embodiments, the pellets are charged to the extruder hopper, which is used to feed the polymerization reactor. In some embodiments, the plastic scrap and catalysts are charged separately into the hopper. In some embodiments, the mixing takes place into the extruder at the time of plastic scrap melting, and the mixture is subsequently fed to the depolymerization reactor.

In some embodiments, the depolymerization reactor is an agitated vessel, operated at temperature ranging from 300° C. to 550° C., alternatively from 350° C. to 500° C., alternatively from 350° C. to 450° C., with an inlet for plastic feedstock and catalyst and an outlet for the gaseous depolymerization product.

In some embodiments and as a result of the depolymerization process, a gaseous stream is generated and sent to a condensation unit, which totally or partially liquifies the stream.

In some embodiments, the condensation section receives effluent gases from the depolymerization reactor and partially condenses the gases into an oily depolymerized product made from or containing hydrocarbons. In some embodiments, a fraction of incondensable gases is collected and stored separately. In some embodiments, the condensation section has one or more stages. In some embodiments, the stages are operated under pressure. In some embodiments, the stages are operated at different temperatures. In some embodiments, the operating conditions of the number of stages, pressure, or temperature permit recovery of the maximum amount of products, according to the volatility of the resulting formed compounds. In some embodiments, the temperature range varies depending on the operating pressure.

In some embodiments, the condensation section has at least two condensation stages. In some embodiments, the condensation stages are operated at descending temperatures. In some embodiments and with small scale equipment, the first condensation stage is operated at a temperature range of 100-120° C. and the second condensation stage is operated at a temperature range of from 2° C. to −20° C.:

In some embodiments, the depolymerization product coming from the condensation stage is subjected to a second depolymerization stage carried out in the presence of the MOF. In some embodiments, the second depolymerization stage is carried out under conditions similar to the first depolymerization stage. In some embodiments, the catalyst and part of the liquid or semiliquid mass are recycled to the first depolymerization reactor from which the solid residue is discharged. In some embodiments, the gaseous effluent is condensed in a subsequent condensation stage under conditions similar to the first depolymerization step.

In some embodiments and at the end of the process, at least 80% wt., alternatively at 90% wt., of the plastic feedstock is converted into a liquid or gaseous depolymerization product.

In some embodiments, the depolymerization product is used as a cracker feedstock. In some embodiments, the depolymerization product is a liquid depolymerization product. In some embodiments, the amount of liquid depolymerization product is higher than 60% wt, alternatively from 65 to 85% wt., of the plastic waste feedstock.

In some embodiments, the liquid depolymerization product is made from or containing a very low amount, of fractions with C28 or higher. In some embodiments, the liquid depolymerization product is free of fractions with C28 or higher. In some embodiments, the liquid depolymerization product is made from or containing an amount of the higher than C28 fraction equal to, or lower than, 4% wt, alternatively lower than 3% wt, alternatively lower than 2% wt, with respect to the total amount of liquid depolymerization product.

In some embodiments, the depolymerization oil has low values of C6-C8 aromatics (Ar) and Branch Index (BI). As used herein, the term “Branch Index (BI)” refers to the molar ratio between internal double bonds with respect to double bond in chain end position (alfa-olefins). In some embodiments, the liquid depolymerization product has a Branch Index no more than 1% wt, alternatively lower than 0.5% wt, alternatively lower than 0.4% wt.

As used herein, the term “C6-C8 aromatics” (Ar) refers to a hydrocarbon with sigma bonds and delocalized pi electrons between carbon atoms forming a circle, wherein total of 6 to 8 carbon atoms are present. In some embodiments, the amount of C6-C8 aromatics present into the liquid depolymerization product is lower than 1.3% wt.

In some embodiments, the catalyst is made from or containing a metal framework oxide (MOF).

In some embodiments, MOFs are highly porous crystalline materials, made from or containing organic linkers and inorganic blocks joined together in a textured structure. In some embodiments, MOFs, having 3D (cage) or 2D (layered) framework type, are used as catalysts.

In some embodiments, the MOF catalyst is selected from the group consisting of MOF including topology, for example, MOFs with zeolitic topologies, such as zeolitic imidazolate frameworks (ZIFs) and zeolite-like metal-organic frameworks (ZMOFs).

In some embodiments, the MOF catalysts have the organic linker selected from the group consisting of carboxylates, phosphonates, N-based groups and N—O-containing groups. In some embodiments, the organic linker is a carboxylate group. In some embodiments, the carboxylate organic linkers are selected from the group consisting of fumaric carboxylate (FA), benzene-1,4-dicarboxylate (BDC), 1,3,5-benzene tricarboxylate (BTC), and formic carboxylate.

In some embodiments, the MOF is selected from the group consisting of Zr-MOF-808 (Zr based BTC linker), Ce-MOF-808 (Ce based BTC+FA linker), MOF-199 (Cu based, BTC linker), and MIL-88A (Fc₂O₃ based, fumaric carboxylate ligand). In some embodiments, the MOF has the formula of Zr₆O₄(OH)₄(—CO₂)₆(BTC)₂(HCOO)₆ for MOF-808; Zn₄O(BDC)₃ for MOF-5, or Cu₃(C₉H₃O₆)₂ for MOF-199.

In some embodiments, the MOFs are selected from the group consisting of IRMOFs series (MOF-5), MOF-74 series, Sandia Metal-Organic Frameworks (SMOFs) series, and zeolitic imidazolate framework (ZIF) series.

In some embodiments, the ZIF series are selected from the group consisting of ZIF-4, ZIF-5, ZIF-6, ZIF-8, ZIF-10, and ZIF-11.

In some embodiments, the ZIF has a topology defined by the metal cations that is identical to the zeolitic framework type SOD or RHO. As used herein, SOD is a three letter framework type code for a sodalite structure type. As used herein, RHO is a three letter framework type code, as defined by the Structure Commission of the International Zeolite Association in the “Atlas of zeolite framework types,” Ch Baerlocher, L B McCusker, and D H Olson, Sixth Revised Edition, Elsevier Amsterdam, 2007. In some embodiments, the ZIF is selected from the group consisting of ZIF-4, ZIF-5, ZIF-8, ZIF-9, ZIF-10, ZIF-11, ZIF-12, ZIF-65, ZIF-67, ZIF-68, ZIF-69, ZIF-70, ZIF-71, ZIF-76, ZIF-78, ZIF-90, ZIF-95, and ZIF-100. In some embodiments, ZIF-8 is commercially available as Basolite® Z1200.

In some embodiments, the MOF has a highly porous coordination framework, HKUST-1. In some embodiments, the framework of HKUST-1 is [Cu₃(benzene-1,3,5-carboxylate)₂] or [Cu₃(BTC)₂]. In some embodiments, [Cu₃(BTC)₂] is referred to as MOF-199 or Cu-BTC. In some embodiments, [Cu₃(BTC)₂] is commercially available as Basolite™ C300 from Sigma Aldrich. In some embodiments, the MOF is crystalline. In some embodiments, the MOF has interconnected [Cu₂(O₂CR)₄] units (where R has an optionally substituted aromatic ring), which create a three-dimensional system of channels with a pore size of 1 nanometer and an accessible porosity of about 40 percent in the solid.

In some embodiments, the MOF is MOF-5, which is Zn₄O(1,4-benzodicarboxylate)₃.

In some embodiments, a poison-suppressing agent is used in association with the catalyst. In some embodiments, the poison-suppressing agent is selected from the group consisting of Ca(OH)₂, Mg(OH)₂, Ba(OH)₂, Sr(OH)₂, CaO, Al₂O₃, and Zr(HPO₄)₂. In some embodiments, the poison-suppressing agent is Zr(HPO₄)₂.

### Characterization

The properties are determined according to the following methods.

### Analytical Methods

Characterization of liquid products: The liquid products from the two traps were characterized by Gas Chromatography (GC) and proton NMR (¹H NMR).

The GC analysis of the liquid product was performed using an Agilent 7890 GC (Agilent Technologies, Santa Clara, CA) equipped with a non-polar column and a flame ionization detector. For the GC data, the weight percent for x<nC7, nC7<x<nC11, nC12<x<nC28, and x>C28 were used to characterize the liquid product.

NMR data were used to characterize the percent of aromatic protons, paraffinic protons and olefinic protons in the liquid product. The examples were analyzed with an addition of CDCl₃ (0.6 g of depolymerize polymer/metal oxide mixture with 0.4 g of CDCl₃). The data were collected on a Bruker AV500 MHz NMR spectrometer (Bruker Corporation, Billerica, MA) at 25° C. with a 5 mm Prodigy probe. One dimension 1H NMR data were processed using TOPSPIN® software (Bruker) with an exponential line broadening window function. Quantitative measurements were performed with a 15 second relaxation delay, a 30° flip angle pulse, and 32 scans, thereby facilitating integrals. The spectral integrations for aromatic olefinic, and paraffinic protons were obtained and used to quantify relative ratios.

### Determination of Fe, Zr, Cu and Zn

The determination of Fe, Zr, Cu and Zn content in the solid catalyst component was carried out via inductively coupled plasma emission spectroscopy on “I.C.P Spectrometer ARL Accuris” or, alternatively, on “I.C.P Spectrometer Icap 7000”. The sample was prepared by analytically weighing, in a “Fluxy” platinum crucible”, 0.1÷0.3 grams of catalyst and 2 grams of lithium metaborate/tetraborate 1/1 mixture. After addition of some drops of KI solution, the crucible was inserted in a “Claisse Fluxy” for the complete burning. The residue was collected with a 5% v/v HNO₃ solution and then analyzed via ICP at the following wavelengths: iron, 259.94 nm/261.187 nm; zirconium 349.62 nm/343.82 nm; copper 327.40 nm; and zinc 213.86 nm.

### EXAMPLES

### Depolymerization Procedure

### Procedure for Depolymerization Test in a 500 ml Round Glass Reactor

30 g of the polymer plastic were loaded in a 500 mL round glass reactor, having three necks equipped with thermocouple and nitrogen inlet. In some instances, polymer plastic was a virgin resin obtained directly from the polyolefin production plants (examples 1˜4 and comparative example 1) or real plastic wastes (rpw) from municipal collection previously sorted (Examples 5-7 and comparative examples 2-3).

The real plastic waste was analyzed and determined to be made from or containing about 97 wt % of polyolefin, wherein the PP/PE ratio was about 30/70), with the residual containing traces of other polymers (PET, PS, PA, PU, or combinations thereof) and inorganic contaminants.

The solid catalyst (2.5 wt % with respect to plastics) was then introduced into the glass reactor. A blank test without any catalyst was performed. Two glass condensers were connected in series and kept at 110° C. and −8° C., respectively, using an oil bath (Cryostat Julabo). The reactor was placed in electrically heating system (mantle bath). The temperature was raised up to 450° C. The pyrolysis process took place. The following experimental parameters were recorded:
L %, sum of the yield of liquid condensable at 110° C.+liquid condensable at −8° C. (with respect to the polymer charged)
S %, yield of solid/waxy residue in the reactor, excluding catalyst (with respect to the polymer charged)
G %, yield in gaseous products not condensable in both condensers (with respect to the polymer charged)

The results are reported in Tables 1 and 2.

### Comparative Example 1

A depolymerization run was carried out using virgin polypropylene as a depolymerization feedstock and without using a depolymerization catalyst. The results are reported in Table 1.

### Example 1

### Preparation of MIL-88A

MIL-88A (Fe₂O₃ based, fumaric carboxylate ligand) was prepared as described in Applied Catalysis B: Environmental 221 (2018), 119-128 and literature cited therein. The resulting compound was characterized by X-Ray analysis (pattern in agreement with literature data) and elemental analysis (Fe=21.3% wt.).

The resulting catalyst was used in a depolymerization run carried out using virgin polypropylene as a depolymerization feedstock. The results are reported in Table 1.

### Example 2

### Preparation of MOF 808

MOF 808 (Zr based BTC linker) was prepared as described in Material Letters 160 (2015), 412-414. The resulting compound was characterized by X-Ray analysis (pattern in agreement with literature data) and elemental analysis (Zr=19.0% wt.).

The resulting catalyst was used in a depolymerization run carried out using virgin polypropylene as a depolymerization feedstock. The results are reported in Table 1.

### Example 3

### Preparation of MOF-199

MOF-199 (Cu based, BTC linker) was prepared as described in Tetrahedron 64 (2008), 8553-8557. The resulting compound was characterized by elemental analysis (Cu=17.6% wt.).

The resulting catalyst was used in a depolymerization run carried out using virgin polypropylene as a depolymerization feedstock. The results are reported in Table 1.

### Example 4

### Preparation of MOF-5

MOF-5 (Zn based, terephthalic acid linker) was prepared as described in Tetrahedron 64 (2008), 8553-8557. A slight modification of the preparation cited there provided in the work-up step for a solvent exchange of dimethyl formamide first with ethanol and subsequently with acetone in place of chloroform. The resulting compound was characterized by elemental analysis.

The resulting catalyst was used in a depolymerization run carried out using virgin polypropylene as a depolymerization feedstock. The results are reported in Table 1.

### Comparative Example 2

A depolymerization run was carried out using real plastic waste as a depolymerization feedstock and without using a depolymerization catalyst. The results are reported in Table 2.

### Example 5

The same catalyst employed in example 2 was used in a depolymerization run carried out using real plastic waste as a depolymerization feedstock. In addition to the catalyst and as a poison suppressing agent, Zr(HPO₄)₂ (2.5% wt with respect to plastic waste) was used. The results are reported in Table 2.

### Example 6

### Preparation of MOF 808-Cerium Doped

Ce-MOF-808-1.5-20FA was prepared according as described in Microporous and Mesoporous Materials 324 (2021), 111303. The resulting compound was characterized by X-Ray analysis (pattern in agreement with literature data).

The resulting was used in a depolymerization run carried out using real plastic waste as a depolymerization feedstock. The results are reported in Table 2.

### Example 7

The depolymerization run was carried out as described in example 6, excepting that, in addition to the catalyst and as a poison suppressing agent, Zr(HPO₄)₂ (2.5% wt with respect to plastic waste) was used. The results are reported in Table 2.

### Comparative Example 3

The depolymerization run was carried out as described in example 5, excepting that Zeolite HY was used instead of MOF-808. The results are reported in Table 2.

TABLE 1

| | | | | | | | 98° C. < | 203° C. < | |
|---|---|---|---|---|---|---|---|---|---|
| | | | | | | BP < | BP < | BP < | BP > |
| | Feed | | S. | L. | G. | 98° C. | 203° C. | 434° C. | 434° C. |
| Ex # | type | Catal type | wt % | wt % | wt % | wt % | wt % | wt % | wt % |
| Comp. 1 | PP | No | 7 | 73 | 20 | 2.4 | 45.0 | 51.0 | 1.6 |
| 1 | PP | MIL-88A | 3 | 76 | 21 | 2.9 | 48.0 | 48.0 | 1.4 |
| 2 | PP | MOF-808 (Zr) | 0 | 84 | 16 | 6.0 | 49.0 | 42.0 | 3.0 |
| 3 | PP | MOF-199 | 2 | 81 | 17 | 3.4 | 46.0 | 50.0 | 1.1 |
| 4 | PP | MOF-5 | 2 | 82 | 16 | 3.5 | 41.0 | 54.0 | 2.2 |

TABLE 2

| | | | | | | | 98° C. < | 203° C. < | | | |
|---|---|---|---|---|---|---|---|---|---|---|---|
| | | | | | | BP < | BP < | BP < | BP > | | |
| | Feed | | S. | L. | G. | 98° C. | 203° C. | 434° C. | 434° C. | BI | Ar |
| Ex # | type | Catalyst type | wt % | wt % | wt % | wt % | wt % | wt % | wt % | % wt | % wt |
| C2 | Rpw | no | 29 | 56 | 15 | 2.0 | 51.0 | 47.0 | 0.0 | — | — |
| 5 | Rpw | MOF-808 (Zr) + Zr(HPO₄)₂ | 21 | 60 | 19 | 1.4 | 31.0 | 66.0 | 1.0 | 0.37 | 1.0 |
| 6 | Rpw | MOF-808 (Ce) | 23 | 59 | 18 | 2.0 | 41.0 | 56.0 | 1.0 | 0.19 | 1.0 |
| 7 | Rpw | MOF-808 (Ce) + Zr(HPO₄)₂ | 11 | 68 | 21 | 1.0 | 31.0 | 67.0 | 1.0 | 0.34 | 1.2 |
| C3 | Rpw | ZeoliteHY + Zr(HPO₄)₂ | 10 | 70 | 20 | 1.0 | 56.0 | 40.0 | 3.0 | 0.83 | 1.3 |

BI = Branching Index
Ar = Aromatics content

What is claimed is:

1. A process for depolymerizing plastics, comprising the steps of:
a) providing a melt plastic waste feedstock comprising recycled polypropylene; and
b) subjecting the melt product obtained in (a) to a temperature ranging from 280° C. to 600° C., thereby obtaining a depolymerization product;
wherein the melt product, the and depolymerization product, or both are contacted with a metal organic framework catalyst.

2. The process according to claim 1, wherein the amount of catalyst ranges from 0.1-20 wt. %, with respect to the total weight of plastic waste feedstock and catalyst.

3. The process according to claim 1, wherein the plastic waste feedstock comprises a mixture of polyethylene and polypropylene in a weight ratio 85:15 to 15:85.

4. The process according to claim 1, wherein the MOF has a 3D (cage) framework.

5. The process according to claim 1, wherein the MOF has a 2D (layered) framework.

6. The process according to claim 1, wherein the MOF catalyst is selected from the group consisting of MOFs with zeolitic topologies.

7. The process according to claim 1, wherein the organic linker of the MOF is selected from the group consisting of carboxylates, phosphonates, N-based groups and N—O-containing groups.

8. The process according to claim 7, wherein the organic linkers are selected from the group consisting of fumaric carboxylate (FA), benzene-1,4-dicarboxylate (BDC), 1,3,5-benzene tricarboxylate (BTC), and formic carboxylate.

9. The process according to claim 8, wherein in which the MOF is selected from the group consisting of Zr-MOF-808 (Zr based BTC linker), Ce-MOF-808 (Ce based BTC+FA linker), MOF-199 (Cu based, BTC linker), and MIL-88A (Fe₂O₃ based, fumaric carboxylate ligand).

10. The process according to claim 1, wherein the MOF is MOF-5, based on Zn₄O(1,4-benzodicarboxylate)₃.

11. The process according to claim 1, wherein the MOF is used in association with a poison suppressing agent.

12. The process according to claim 11, wherein the poison suppressing agent is selected from the group consisting of Ca(OH)₂, Mg(OH)₂, Ba(OH)₂, Sr(OH)₂, CaO, Al₂O₃, and Zr(HPO₄)₂.

13. The process according to claim 1, wherein the amount of liquid depolymerization product is higher than 60% wt. of the plastic waste feedstock.

14. The process according to claim 1, wherein the amount of the higher than C28 fraction in the liquid depolymerization product is equal to, or lower than, 4%, with respect to the total amount of liquid depolymerization product.

15. The process according to claim 1, wherein the Branch Index of the liquid depolymerization product is no more than 1% wt.
